(19) United States
(12) Patent Application Publication
(10) Pub. No.: US 2004/0143222 A1
(43) Pub. Date: Jul. 22, 2004

(54) Dental anesthetic injection apparatus and methods for administering dental injections

(76) Inventor: Ronald P. Spinello, Red Lion, PA (US)

Correspondence Address: GALGANO & BURKE, Suite 135, 300 Rabro Drive, Hauppauge, NY, 11788, US

(21) Appl. No.: 10/664,380

(22) Filed: Sep. 17, 2003

Related U.S. Application Data
(63) Continuation-in-part of application No. 10/347,668, filed on Jan. 17, 2003.

(51) Int. Cl. A61M 5/00

(52) U.S. Cl. 604/181; 222/325

(57) ABSTRACT

Dental anesthetic injection apparatus and methods for administering dental injections are disclosed. One embodiment comprises a portable device comprising a foot pedal, a control unit and a delivery head which receives a disposable cartridge of dental anesthetic and connects with a disposable syringe kit. The portability of this unit facilitates placing the dental cartridge within the dentist's field of vision while administering an injection. This and other embodiments provide a pen-grip type of needle grip which is movable independently of the anesthetic cartridge, and a programmable flow rate. Other embodiments comprise delivery heads mountable on dental equipment or fixtures, such as the dental chair.

### RELATED APPLICATION DATA

This application is a continuation-in-part of U.S. patent application Ser. No. 10/347,668 filed Jan. 17, 2003.

The present invention is directed to anesthetic injection apparatus and methods and, more particularly, to dental anesthetic injection apparatus and methods for administering dental injections.

### BACKGROUND OF INVENTION

The use of local anesthesia in dentistry is well documented and has been in use for many years. Although its use has made modern dental treatment possible, it has been reported that more than 50% of the adult patients fear injections, and certain injections have traditionally been associated with a significant degree of discomfort. It is ironic that local anesthesia is both the salvation and the bane of modern dentistry.

In recent years, the use of computerized local anesthesia delivery was introduced. Two U.S. patents were awarded to the inventor of the present invention namely U.S. Pat. No. 4,747,824 entitled Hypodermic Anesthetic Injection Method and U.S. Pat. No. 5,180,371 entitled Hypodermic Anesthetic Injection Apparatus and Method. A product embodying these inventions has been marketed under the trademark Wand®. Another injection unit was disclosed in U.S. Pat. No. 6,022,337 entitled Dental Anesthetic and Delivery Injection Unit. Early efforts in the development of this technology were implemented by the Wand®, and make use of two discrete flow rates, a slow speed, and a high speed, with means to switch between the two. The slow speed is used to allow the tissues to slowly absorb the anesthetic's numbing effect. The high speed is used to facilitate the injection. The decision as to when to change flow rates was left to the judgement of the operator who selectively chose between the slow and high-speed flow rates by means of a two-position ancillary foot pedal.

Another approach to this problem invented by the present inventor is disclosed in U.S. Pat. No. 6,113,574 entitled Anesthetic Injection Apparatus and Methods. This invention makes use of a system in which anesthetic is delivered automatically at a gradually increasing rate, which is carefully programmed to anesthetize the local tissues before pain sensation is perceived. This novel “ramp flow” feature allows for an injection that is below the patient's ability to sense pain. The injection is administered without the burden of switching between speeds with a foot pedal switch, and with no unnecessary delay. Further, this improved delivery system provides a built in pressure sensor that indicates to the operator when optimal pressure is experienced. This feature was not envisioned by earlier work with the Wand®, but is particularly desirable in complicated injection procedures such as the periodontal ligament injection (PDL).

Needle insertion is seldom a cause of significant pain in dental injections, but lateral movement of the needle in the tissues does cause pain. With a conventional syringe any movement on the part of the operator or patient is antagonistic. A common feature which can be used by all of the above referenced inventions is the pen grip syringe device. With the pen grip syringe device, a fulcrum or resting point can be established in the mouth close to the injection site. Any movement on the part of the operator or patient then becomes collateral. The pen grip affords delicate, precise needle insertion and stabilization. In addition, the pen grip delivery system offers advantages for the psychological elements of a dental injection. It does not resemble a syringe, is much smaller and is believed to appear less “threatening”. Anxiety is reduced and patient confidence is increased, desired elements in successful dental treatments. This is true with all patients, and especially children and needle phobics. Focus group studies strongly indicate that the most significant perceived advantage to the dentist of the new computer assisted technology is the ability to provide a dental injection using a pen grip syringe device.

These advantages notwithstanding, much resistance to the above-described technology has been experienced in the market place. The dentist is accustomed to being in total control of the injection. Inherent in this control is the dentist's ability to maintain visual contact with the cartridge of anesthetic, thus being assured of the flow rate, the volume (dosage) of the anesthetic solution dispensed, and the aspiration function during the administration of the injection procedure. Also inherent in the control is the ability of the dentist to accelerate the flow rate independent of any pre-programmed computer control when desired. Most products which have been introduced using the above technology have provided a dispensing unit that is remote from the operator, usually on a counter behind the patient. Information regarding the flow rate, volume, and aspirating cycle is translated to the dentist by means of lights and sound indicators, rather than by direct visualization of the cartridge. This is often perceived as loss of control by the dentist, and has resulted in significant market resistance to the products.

A second objection to the above-described technology as it presently exists is the use of a designated foot pedal for operation. Dentists typically have an air operated foot pedal in their treatment rooms. Such foot pedals are used to operate most of their treatment tools, such as the dental drill and related instruments. Newly introduced equipment; for example the intra-oral camera, laser instruments, endodontic equipment and ultrasonic cleaning instruments all come with designated foot pedals. The treatment room has thus become a complexity of foot pedals and related connecting cords, much to the chagrin of the dentist. The need to add ancillary foot pedals in the treatment room discourages their purchase by some dentists.

A third complaint regarding the known technology is that the time of the injection is too slow, and the dentist has no way of accelerating beyond the computer-controlled limitations set by the device. This is perceived as loss of control by the dentist, a waste of chair time, and an unusually prolonged injection procedure for the patient. This complaint, together with the other cited objections, has resulted in significant market resistance to the product.

One attempt to solve these problems has been called the CCS (computer controlled syringe) and is described in U.S. Pat. No. 5,690,618 entitled Electronic Syringe. This product places the motor drive and the cartridge of anesthetic, as well as all other controls, in a handpiece, thus allowing the dentist direct visibility of the anesthetic cartridge during an injection, and control of the injection without the need for a foot pedal. Ironically, by placing the cartridge and controls in the handpiece, this product eliminates a principal advantage of the technology, namely the pen grip syringe device. Some dentists believe the size and shape of the CCS instrument is not ergonomically correct, appears more threatening to patients, and is more difficult to use than a conventional syringe.

Many dentists have a preference for certain types of disposable needles. For example, the use of disposable double-ended needles is common in dentistry. Some dentists prefer longer or shorter needles for certain types of injections. It would, therefore, be desirable to provide injection apparatus and/or disposable syringe kits which permit a dentist to utilize his choice of disposable needle.

### SUMMARY OF THE INVENTION

Various embodiments of the present invention comprise computer assisted anesthetic delivery systems with the advantages of a pen grip syringe device and the unique advantage of placing the cartridge of anesthetic proximate the field of vision of the dentist during the injection procedure. One embodiment of the present invention also provides a gradually ramped, variable injection flow rate. This allows the dentist to accelerate or decelerate the flow rate at any time during the injection, thus putting the dentist in total control of the injection flow rate.

According to an alternative embodiment, the anesthetic is delivered automatically at a rapidly increasing flow rate, programmed to accelerate from a slow to a rapid flow rate over a relatively short time period, for example, five seconds. According to this embodiment, once a rapid flow rate is established, the flow rate is maintained until the injection is completed. The entire injection may take as little as ten seconds which is about as fast as is capable by traditionally hand-held syringes. Moreover, at any time during the injection, the dentist can interrupt the anesthetic administration by removing his/her foot from the foot pedal. This stops the injection.

When the foot pedal is reactivated, the ramp cycle begins again from the beginning. In this way, the dentist may restart the cycle as often as is necessary for patient comfort. The dentist thus has the choice: a slow and gradual series of ramps for enhanced pain control during the injection, or a rapid advance to high speed flow rate, to facilitate a rapid injection, for example, when the patient is partially anesthetized, and increased dosage of the anesthetic solution is indicated.

One preferred embodiment of the present invention comprises a self-contained unit which is lightweight and completely portable. The foot pedal control of this embodiment is an integral part of the unit and does not require connecting cables. The device is powered by a rechargeable battery and, therefore, does not even require an electrical cord during office hours. The battery can simply be recharged overnight or over a weekend. The portability of this unit facilitates optimum positioning for each dentist-patient combination and permits the positioning of the cartridge proximate to the dentist's line of sight during that injection. The height of the cartridge is also preferably adjustable.

Another aspect of the present invention which may be incorporated into various embodiments when desired comprises a pressure sensor that indicates to the operator when optimal pressure is experienced. This is accomplished electronically by sensing a current change corresponding to a predetermined load on the motor. The load condition is communicated to the operator by a signal, e.g. a blinking light. If the load on the motor exceeds some predetermined criteria, this too is communicated to the operator by another signal, e.g. a steady red light. This would occur, for example, when a blockage of the flow of anesthetic solution through the needle occurs. The operator would be alerted to this fact and take necessary steps to eliminate the blockage. This pressure-sensing feature is desirable in complicated injection procedures such as the periodontal ligament injection (PDL) where significant resistance to the deposition of the anesthetic is a necessary indicator for a successful injection. Inability to sense pressure frequently results in failure to obtain anesthesia. More importantly, failure to sense pressure can result in improper positioning of the needle into the soft tissue resulting in tissue sloughing. During clinical trials, the device with its pressure sensing capability has resulted in a high degree of success, for the indicated PDL injection procedures. The ability to anesthetize a single tooth predictably, with immediate onset, and with no concomitant lip, cheek and tongue numbness has obvious and proven advantages to both the dentist and the patient. This aspect of the invention can be used to administer all traditional infiltrations and block injections in either arch. In addition to the unique PDL injection, the invention also facilitates the newly discovered anterior middle superior alveolar (AMSA) nerve block.

Another embodiment provides operation of the device using an existing air operated foot control commonly found in dental treatment rooms. This embodiment still allows the operator to maintain direct visual contact with the cartridge of anesthetic solution during the administration of the injection.

As an added benefit of the invention, the length of microbore tubing required, usually about 4.5 feet in the case of the Wand®, can be significantly reduced, e.g., to as little as 1.5 feet, at a significant cost savings. This is a result of the unique positioning of the delivery device (remote unit), preferably to within inches of the patient's mouth. Furthermore, the use of shorter tubing results in less waste of anesthetic fluid. Those skilled in the art will appreciate that it is necessary to void the air from the tubing prior to injecting in order to eliminate the possibility of introducing air into the tissues. The amount of wasted residual fluid, which can be about 0.3 ml in a 4.5 length of tubing, can be reduced considerably using the shorter length of tubing of the present invention.

One embodiment of the present invention comprises two distinct devices, a control unit and a remote unit. The control unit preferably comprises a motor with computer controls, a worm gear, and a hydraulic piston. This control unit is preferably placed beneath the dental chair where it is connected to electrical power and an air output from an existing air controlled foot pedal. This unit has two inputs from the foot pedal. When gentle pressure is applied to the pedal by the operator, the motor is activated to promote a preprogrammed ramp up injection flow-rate, as taught by the present inventor's U.S. Pat. No. 6,113,574. When additional foot pressure is brought to bear on the foot pedal the motor turns at top speed, thereby facilitating a rapid flow rate. Thus the operator can selectively choose between operating modes. The other functions of the assembly can be disclosed in U.S. Pat. No. 6,113,574.

The remote unit on this embodiment is connected to the control unit electrically and hydraulically by an umbilical chord. The remote unit of this embodiment is preferably fastened to an existing dental chair, e.g. on the side preferably adjacent to the patient's head. The remote unit can be provided with an extension hinge with a switch. When in the folded position, the unit is tucked in close to the side of the chair, out of the way. In this position the foot control is electrically turned off so that the foot pedal can be used for its other purposes in the dental treatment room. When desired, the remote unit is unfolded to a position proximate the patient where it is in direct view of the dentist, ready for a dental injection.

The remote unit uses a disposable syringe consisting of a cartridge holster, and pen grip syringe device, such as the one described in the present inventor's U.S. Pat. No. 6,296,623. As explained in that patent, a cartridge of anesthetic is inserted into the cartridge holster, and the holster is inserted into the Remote Unit. Rotation of the cartridge holster locks the holster in position, and activates a switch that “primes” the Control Unit. A plunger extends from the hydraulic piston, and serves to expel the liquid anesthetic solution from the cartridge, through the microbore tubing, and pen grip syringe device with attached needle, and into the patient, all as described in the present inventor's prior patents (U.S. Pat. Nos. 6,113,574 and 6,296,623).

Alternatively, a Remote Unit is provided which makes use of a hinged capsule that becomes an integral part of the housing. When opened, the anesthetic cartridge is placed passively into a cradle. When the hinged door is closed, a switch is activated to prime the Control unit. The plunger forces the cartridge onto the plastic insert, causing penetration, and then communication of the cartridge fluid with the microbore tubing.

The injection procedure is then completed. If desired, the pressure-sensing feature of U.S. patent (U.S. Pat. No. 6,113,574) can be incorporated into the present invention. In this case, light indicia are positioned on the side of the Remote Unit, in direct vision of the dentist, so that information regarding pressure is clearly visible during the injection procedure. If anesthetic solution remains in the cartridge, the dentist folds the unit into the off position, shutting off the power to the foot pedal. If the dentist wishes to make use of the saved anesthetic solution for a further procedure on the same patient, the dentist may unfold the unit and continue the use of the device. However at the end of the injection procedure, the dentist rotates the cartridge holster, sending a signal to the motor to retract the plunger to the start or “un-primed” position. Another cartridge of anesthetic can be inserted into the holster, if it is to be re-used on the same patient, or the entire disposable syringe kit is discarded according to normal practice. The unit is then ready to receive a new disposable syringe kit for a new patient.

Other uses for the unique, remote positioning of a control unit are envisioned by this invention. For example, a fiber optic light source can be placed in the unit, to illuminate the working end of certain instruments such as cavity probes, periodontal probes, and scaling instruments. Other electronic controlled devices can become part of the system, thereby eliminating ancillary foot pedals. Such instruments could include pulp testers, laser operated decay indicating instruments, and apex locators.

The invention offers advantages for both the physical and psychological elements of a dental injection. It does not resemble a syringe and is not “threatening” in appearance. There is a perception that a computer can perform tasks more accurately than they can be performed manually and some patients find this comforting. Anxiety is reduced and patient confidence is increased. The entire injection experience becomes a more positive one for the operator and the patients, especially children and needle phobic patients.

Other embodiments of the present invention comprise novel needle handles designed for connection with standard disposable double-ended dental needles in a manner which prevents leakage. Dental handles are also disclosed which comprise removable extensions. These aspects of the present invention provide dentists with versatility and the ability to use short needles, long needles, short handles and/or long handles, as desired. Other aspects of the present invention comprise other designs for securing disposable dental cartridges and/or disposable dental cartridge holsters to the delivery head of an injection apparatus. One embodiment comprises a bracket under which a substantial portion of a disposable cartridge holster is positioned. Another embodiment comprises a disposable cartridge holster comprising a polygonal, preferably square, base which is inserted and then rotated into a delivery head for securely connecting the cartridge holster to the delivery head.

### BRIEF DESCRIPTION OF THE DRAWINGS

FIG. 1 illustrates one preferred embodiment of the present invention with portions of a disposable syringe kit in place.

FIGS. 2-4 illustrate possible positions of the device shown in FIG. 1 during administration of an injection.

FIG. 5 is an enlarged, cross-sectional view of the control unit portion of the instrument shown in FIG. 1.

FIG. 5(A) is a cross-sectional view of a control unit portion comprising a height extension.

FIG. 6 is a cross-sectional side view of the control unit shown in FIG. 5.

FIG. 7 is a front view of the delivery head portion of the embodiment shown in FIG. 1.

FIG. 8 is a cross-sectional, side view of the delivery head shown in FIG. 7.

FIGS. 9(A)-(C) illustrate a removable plunger of one embodiment of the present invention.

FIG. 10 is a partially-exploded view of a disposable syringe kit which may be utilized with the present invention.

FIG. 11 illustrates the placement of a needle handle into the needle cap holder of the delivery head.

FIG. 12 illustrates the placement of an anesthetic cartridge into a cartridge holster of the syringe kit.

FIGS. 13 and 14 illustrate the placement of a cartridge holster onto a delivery head.

FIG. 15 illustrates the withdrawal of a needle handle from the needle cap holder.

FIGS. 16(A) and 16(B) illustrate the operation of a foot pedal switch in the dispense and aspiration modes, respectively.

FIG. 17 illustrates the return of the needle handle into the needle cap holder.

FIGS. 18(A)-(C) illustrate alternative embodiments of disposable syringe kits of the present invention.

FIGS. 18(D)-(F) illustrate the connection of the disposable syringe kit of FIG. 18(A) onto separate disposable needles of different lengths.

FIGS. 19(A)-(G) illustrate the positioning of the disposable syringe kit onto the delivery head of the embodiment of the present invention shown in FIG. 18(A).

FIGS. 20 and 21 illustrates portions of cartridge holster, delivery head and disposable syringe kit of an alternative embodiment of the present invention.

FIGS. 22-28 illustrate an alternative delivery head and disposable syringe kit of the present invention.

FIG. 29 illustrates an alternative embodiment of the present invention wherein a delivery unit is connected to a dental chair.

FIG. 30 is a close-up view of the delivery unit showing FIG. 19.

FIGS. 31(A)-(C) illustrate an alternative foot pedal useful with the present invention.

FIG. 32 illustrates an air pressure switch manifold useful with the foot pedal shown in FIGS. 31(A)-(C).

FIG. 33 is a top view of a needle handle of the present invention and one type of standard double-ended dental needle.

FIGS. 34A and 34B are cross-sectional views taken along lines A-A and B-B of FIG. 33.

FIG. 35 is a cross-sectional side view of the needle handle shown in FIG. 33.

FIG. 36 is a cross-sectional top view of the needle handle shown in FIG. 33 with the double-ended dental needle attached.

FIG. 37 is a cross-sectional side view of an alternative needle handle of the present invention and one type of double-ended dental needle.

FIG. 38 is a cross-sectional top view of an alternative embodiment of a needle handle with a double-ended dental needle attached.

FIGS. 39A-D illustrate various shapes of rubber inserts which can be used with the present invention.

FIGS. 40A-F illustrate one embodiment of a needle handle with a removable extension of one embodiment of the present invention.

FIG. 41A is an exploded view of an alternative embodiment of a needle handle of the present invention.

FIGS. 41B and 41C are cross-sectional views taken along line B-B and C-C of FIG. 41A.

FIGS. 42A and 42B illustrate the connection of a double ended dental needle to an assembled needle handle of the type shown in FIG. 41A.

FIG. 42C illustrates another embodiment of an assembled needle handle.

FIGS. 43A-H illustrate front, side and cross-sectional views of an alternative cartridge holster and delivery head of the present invention.

FIGS. 44A-G illustrate an alternative cartridge holster of the present invention.

FIGS. 45A-B and 46A-D illustrate an alternative cartridge holster and delivery head of the present invention.

### DETAILED DESCRIPTION

Various embodiments of the present invention comprise a delivery head adapted to be connected to a disposable syringe kit and to position a cartridge of anesthetic, such as lidocaine, sufficiently proximate the patient's mouth so that the dentist can view the cartridge without turning his head during the administration of an anesthetic injection. The cartridge is preferably positioned comfortably within the dentist's field of vision so that the dentist can view the cartridge without turning his head away from the patient. According to the most preferred embodiments, the positioning of the cartridge of anesthetic is movable relative to the dental chair for maximum adjustability for different patients and/or for moving the delivery head out of the way of the dentist, the dental assistant and the patient when not in use.

Various embodiments of the present invention comprise a delivery head adapted to be connected to a disposable syringe kit and to position a cartridge of anesthetic, such as lidocaine, sufficiently proximate the patient's mouth so that the dentist can view the cartridge without turning his head during the administration of an anesthetic injection. The cartridge is preferably positioned comfortably within the dentist's field of vision so that the dentist can view the cartridge without turning his head away from the patient. According to the most preferred embodiments, the positioning of the cartridge of anesthetic is movable relative to the dental chair for maximum adjustability for different patients and/or for moving the delivery head out of the way of the dentist, the dental assistant and the patient when not in use.

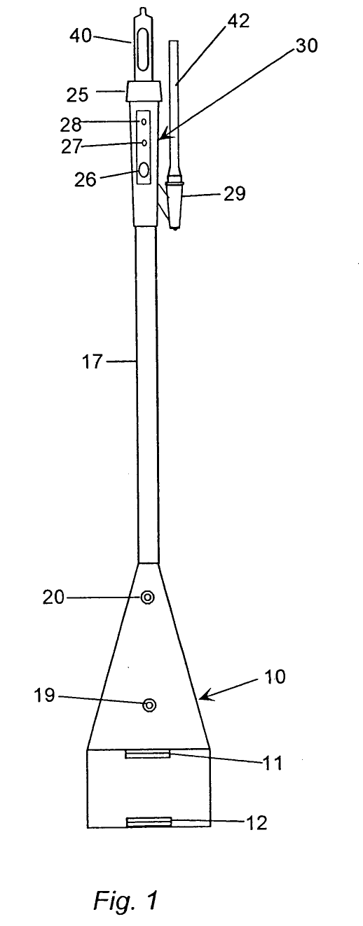

FIG. 1 illustrates a self-contained computer assisted anesthetic injection device according to a preferred embodiment of the present invention. This device comprises a control unit 10 in a base and a delivery head 30. The delivery head is adapted to be connected to a disposable syringe kit adapted to receive a cartridge of anesthetic.

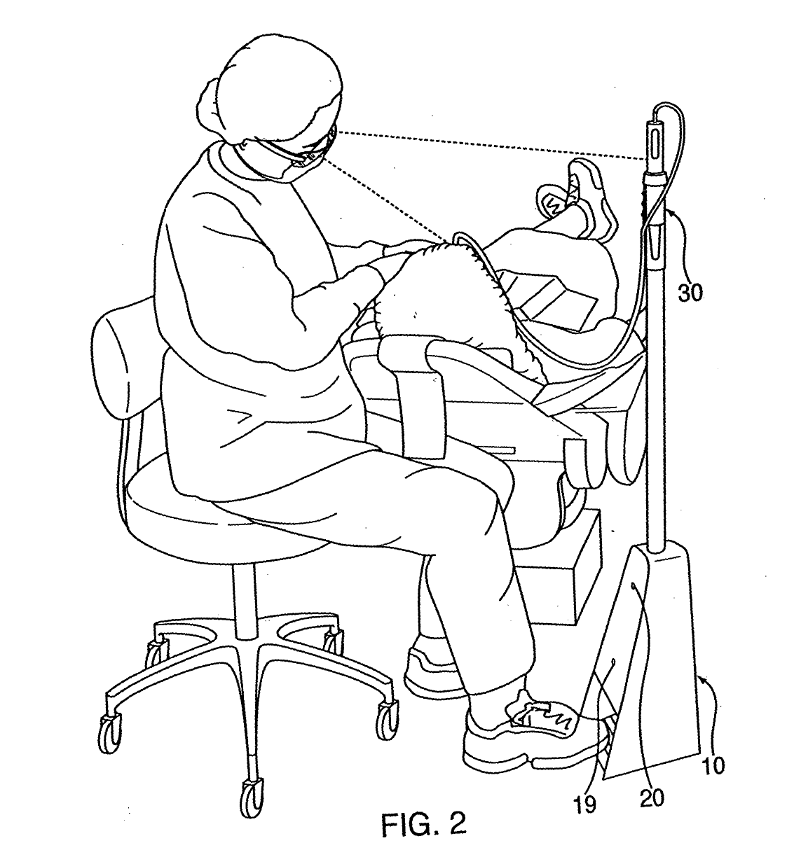

FIG. 2 illustrates one possible position of a dentist utilizing the embodiment of the present invention shown in FIG. 1. According to this embodiment, the dentist is seated with her feet positioned under the dental chair and with one foot positioned to operate the pedals in the control unit 10 in a manner described below.

FIG. 3 is a front view of the dentist and patient shown in FIG. 2 illustrating the positioning of a disposable syringe kit and cartridge of anesthetic within the dentist's field of vision.

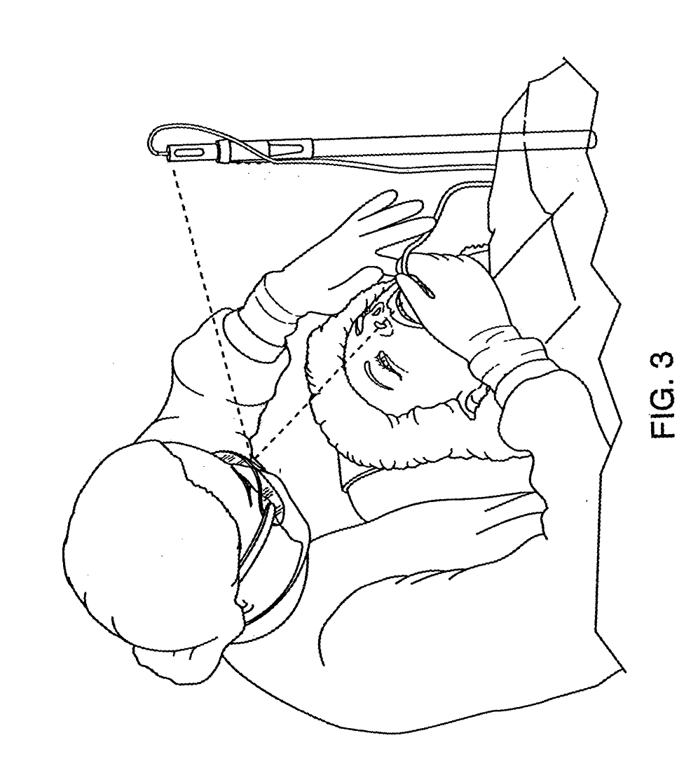

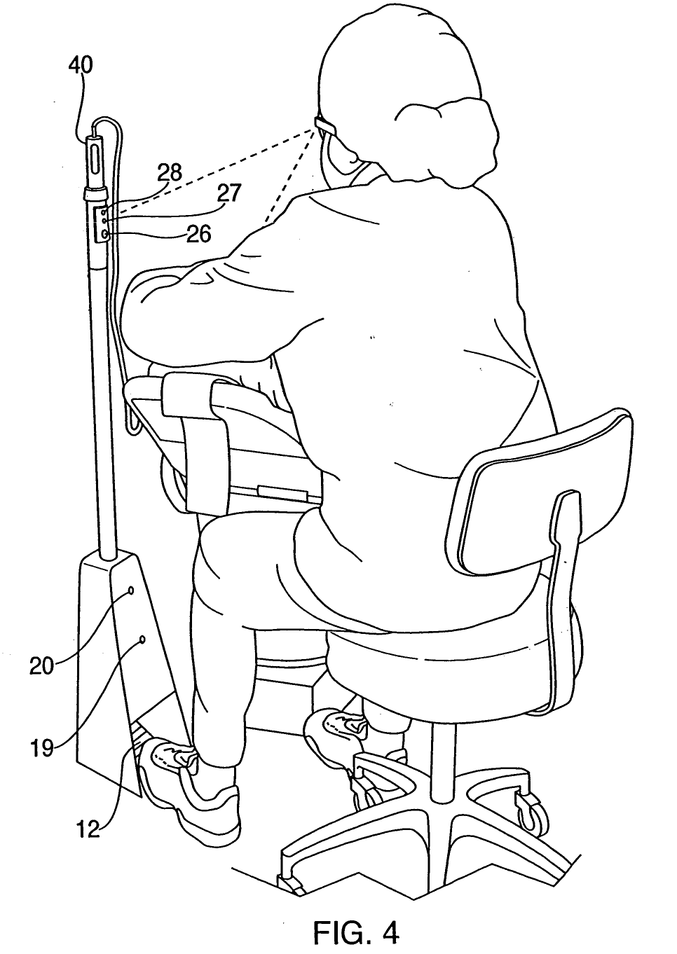

FIG. 4 illustrates a rear view of the position shown in FIG. 3 while administering an injection utilizing the device shown in FIG. 1. A comparison of FIG. 2 with FIGS. 3 and 4 shows that this preferred embodiment of the present invention can advantageously be readily moved to either side of a dentist chair. These figures illustrate a left-handed dentist and a right-handed dentist, respectively. FIG. 3 also illustrates the convenience of positioning provided by the present invention which permits the cartridge to be positioned close to the dentist's line of sight. This permits the dentist to view the cartridge while administering an injection. The cartridge is preferably positioned so that the dentist need not turn her hand significantly, and most preferably, not at all to view both the injection site and the cartridge during an injection. A preferred positioning places the cartridge within a field of view about 50° or less from the injection site as measured from the dentist's eyes.

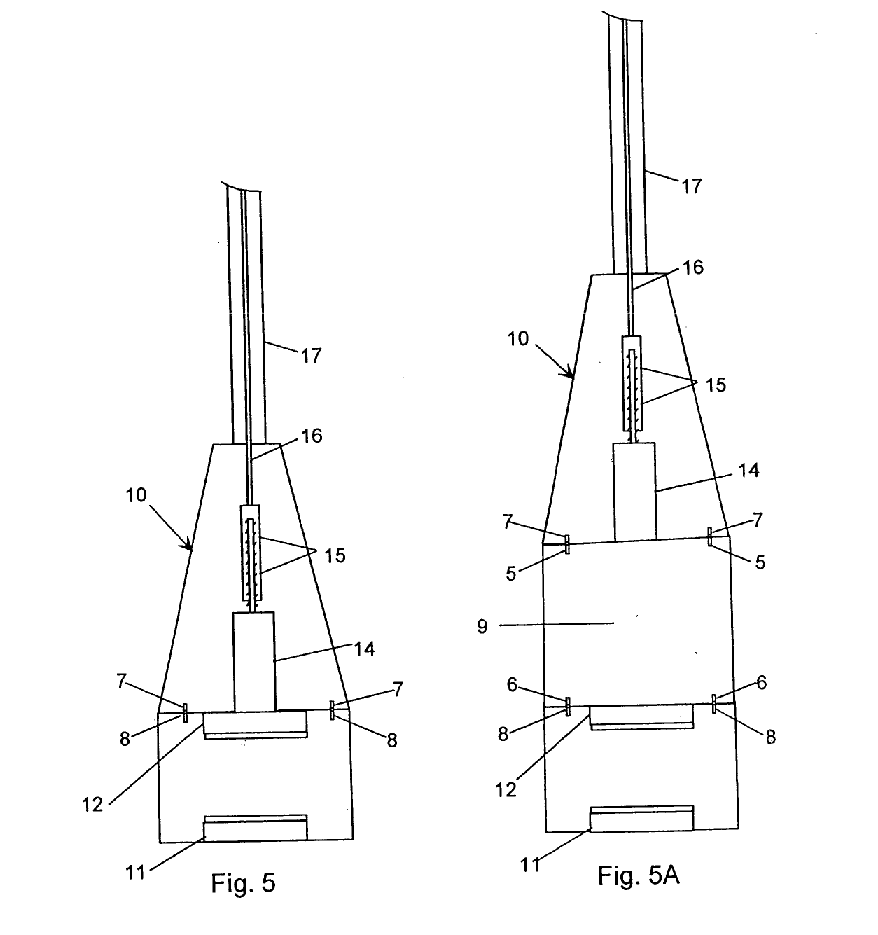

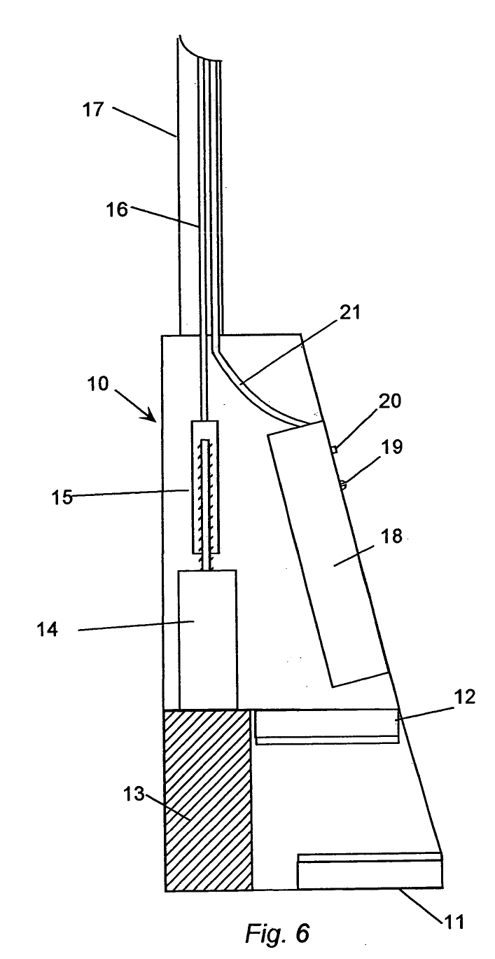

Turning to the structural details of the illustrated embodiments of the present invention, FIGS. 5 and 6 are cross-sectional front and side views, respectively, of the control unit shown in FIG. 1. The illustrated control unit 10 comprises a foot operated injection switch 11, a foot operated aspiration switch 12, a battery pack 13, a motor 14, and a worm gear 15 connected to a connecting rod 16. Connecting rod 16 passes through a conduit 17 for connection to a plunger as described below. The control unit 10 also comprises a microprocessor 18, a battery recharge plug 19, a power switch 20 and delivery head wires 21 which extend to delivery head 30. In this illustrated embodiment, the lower portion of the control unit is designed to receive the forward portion of a dentist's foot. Depression of lower injection switch 11 or the application of foot pressure to upper aspiration switch 12 causes the dispensing and aspiration at the needle tip, respectively, as further described below. The battery pack 13 of this preferred embodiment is preferably rechargeable by connection of a power cord (not shown) to battery recharge plug 19. It is also within the scope of the present invention to eliminate the need for a battery pack and to provide electricity utilizing a plug connectable to an AC outlet. A rechargeable battery is believed to be preferable since it eliminates the need for additional power cords and facilitates the portability of the entire injection device during patient hours. It is also within the scope of the present invention to provide with battery operation and AC operation, as desired by the dentist. Microprocessor 18 controls the rotation of motor 14 which drives the worm gear 15 in order to advance or retract connecting rod 16. The precise connections between the dispense switch 11, aspiration switch 12, battery pack 13, motor 14, microprocessor 18 and other components of this embodiment are not shown in further detail as they are well within the scope of one of ordinary skill in the art.

FIG. 5A illustrates an alternative embodiment of the device shown in FIG. 5 wherein an extension 9 is provided to increase the height of the delivery head 30. Extension 9 can be provided in different heights and can be removable by a dentist. The illustrated extension 9 comprises electrical contacts 5 and 6 which extends electrical communication between contacts 7 and 8.

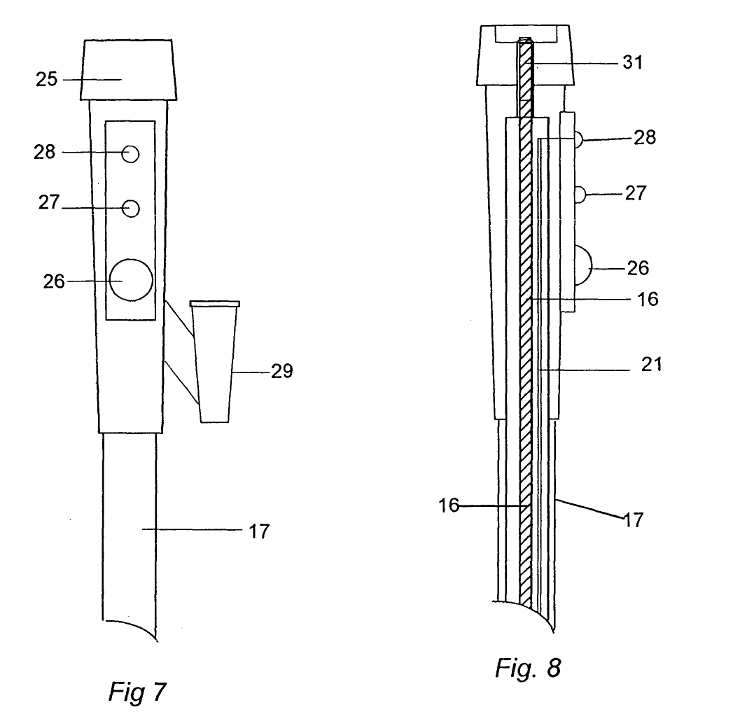

FIGS. 7 and 8 illustrate the delivery head of the device shown in FIG. 1. FIG. 7 illustrates the upper portion of conduit 17, a cartridge holster connector 25, a recycle button 26, a “POWER ON” light 27, a pressure indicator 28 and a needle cap holder 29. In a manner described in further detail below, the pressure indicator 28 preferably provides a plurality of signals to the dentist. Signals can be differentiated by color, by illuminating and extinguishing a single light, or by the rate at which a light flashes. Alternatively, a single pressure indicator 28 can be replaced by a numerical display, such as an LED indicator, or other suitable display device. The power light 27 can be caused to indicate a low battery, e.g. by flashing. The device is preferably provided with a cord and transformer so that it can be plugged into an AC outlet for uninterrupted use if the battery charge becomes depleted.

The cross-sectional view in FIG. 8 illustrates the plunger assembly 31 which is in contact with the upper end of connecting rod 16. The upper end of the electrical wires 21 connect the electrical components from the control unit 10 with the indicator lights 27, 28 and recycle button 26.

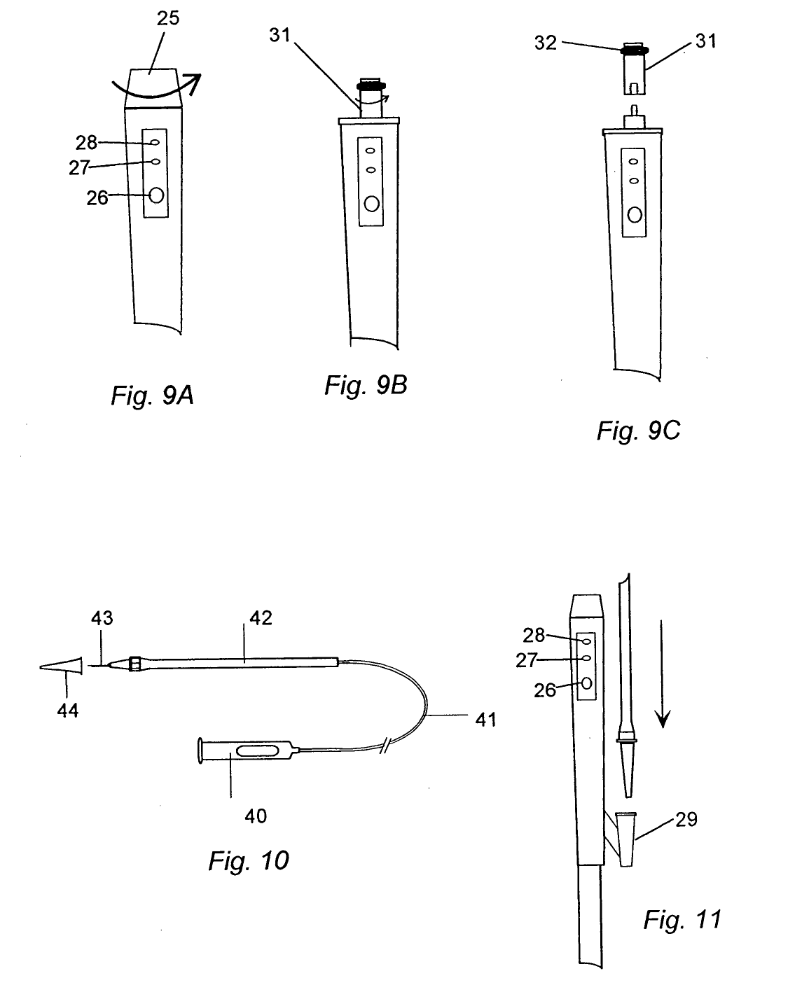

As shown in FIGS. 9(A) through (C), according to this preferred embodiment of the present invention, the cartridge holster connector 25 can be disassembled and a plunger 31 can be removed for replacement or cleaning. According to this illustrated embodiment, cartridge holder connector 25 is simply unscrewed from the top of the delivery head and plunger 31 can also be simply removed by unscrewing. Those skilled in the art will appreciate that on occasion a cartridge may break. If any glass shards remain in this fixture, they can be readily removed and discarded in an appropriate manner. The removable components can be simply rinsed under running water, cleaned and disinfected using standard procedures. FIGS. 9(B) and 9(C) also illustrate an O-ring 32 which creates a negative pressure with the cartridge piston for aspiration. Alternative embodiments described below utilize a barb to retract the cartridge piston during aspiration.

FIG. 10 illustrates a disposable syringe kit of a type which is known in the art comprising a cartridge holster 40 for receiving a cartridge of anesthetic, a length of microbore tubing 41, a needle handle 42, a needle 43 and a protective needle cap 44.

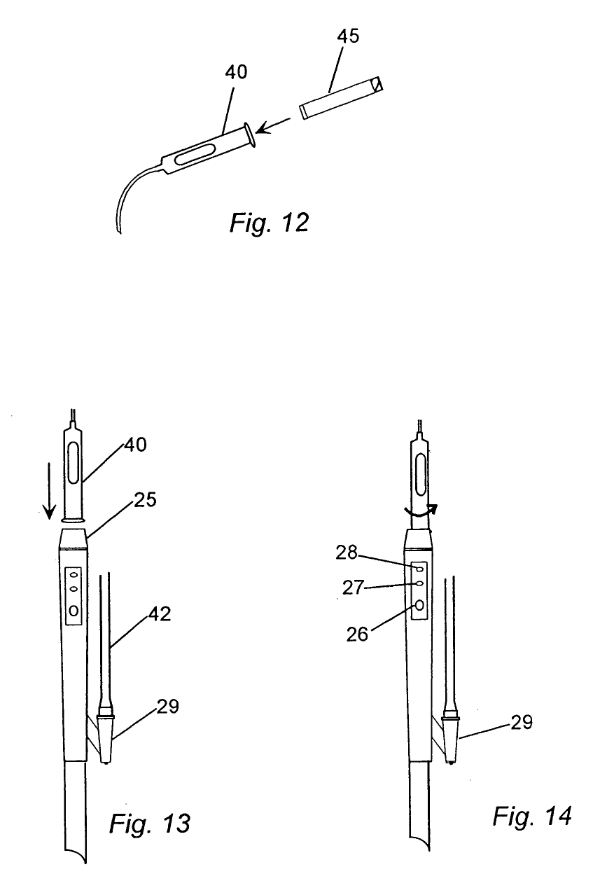

The preferred method of operating the anesthetic injection device shown in FIGS. 1 and 9 will now be described with reference to FIGS. 11 through 17. Initially, the system is turned on via power switch 20 located on the control unit. Then recycle button 26 is depressed to fully retract plunger 31. The disposable syringe kit shown in FIG. 10 is preferably removed from a sterile wrapper. The needle handle 42 with the capped needle 43 is positioned in needle cap holder 29 as shown in FIG. 11. A cartridge of anesthetic 45 is inserted into the cartridge holster 40 as shown in FIG. 12 and the holster 40 is then connected to the cartridge holster connector 25. As shown in FIGS. 13 and 14, the cartridge holster is preferably pressed down into the cartridge connector and rotated. The assembly of the disposable syringe kit can be performed prior to the patient taking a seat in the dental chair or otherwise outside the view of the patient.

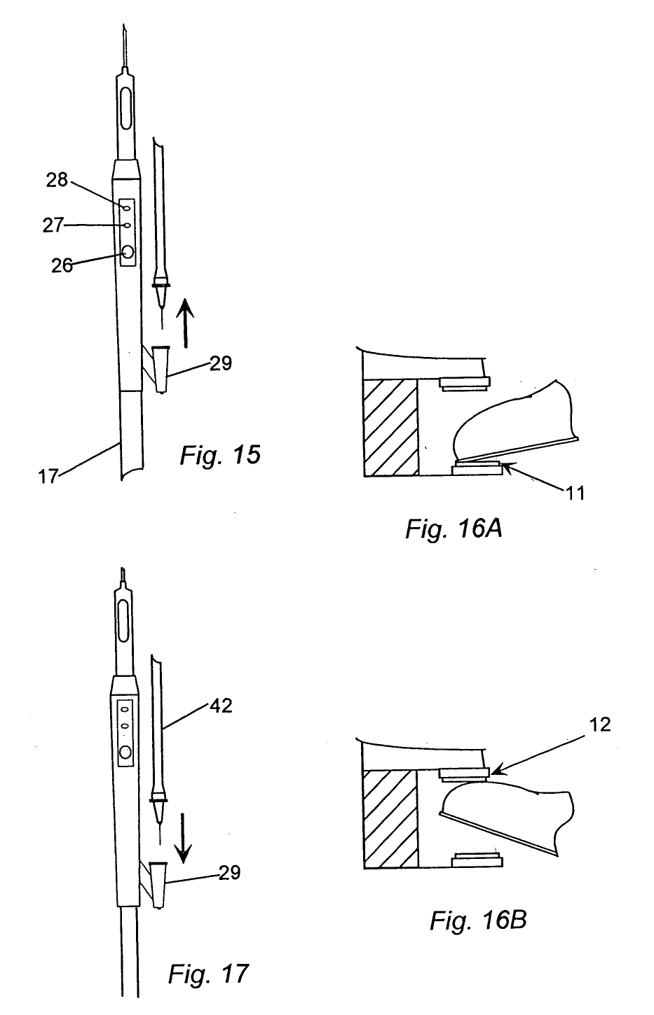

When the patient is ready for the injection, the needle handle 42 is withdrawn from the needle cap holder 29 leaving the protective needle cap 44 in the holder 29 on the delivery head 30 as shown in FIG. 15. The dentist then applies foot pressure to the dispense switch 11 as illustrated in FIG. 16(A). This will advance the plunger 31 and activate a light on flow rate indicator 28. After air has been expelled from the cartridge and the disposable syringe kit, the dentist can insert the needle. Prior to dispensing the desire dosage, the dentist should aspirate by touching the aspiration switch 12 with his foot as shown in FIG. 16(B). This causes the plunger to retract. According to this preferred embodiment of the present invention, when the dentist releases his foot from the aspiration switch, the plunger-advantageously returns to the position it was in immediately prior to aspiration. Following an acceptable-aspiration, the dentist can proceed with administering the anesthetic by depressing dispense switch 11 with his foot. After the desired dosage of anesthesia has been administered, the needle handle 42 and needle 43 are returned to the needle cap holder 29 as shown in FIG. 17.

The portability of this injection device advantageously make all functions of the injection, including the rate of flow, volume of anesthetic dispensed, tissue resistance (pressure) and aspiration cycle, clearly visible to the dentist. The dentist is, therefore, in complete control of the injection, without the need for audio signals, and without the need to turn his/her head away from the patient during the critical phase of the injection procedure.

The flow rate control and pressure are preferably preprogrammed into the microprocessor. The flow rate is preferably variable from extremely slow, to extremely fast, all at the discretion of the dentist. The flow rate is controlled by the foot control switch 11 in a manner similar to controls of other instruments commonly used in dentistry, e.g., greater foot pressure resulting in a higher flow rate. The flow rate transitions are gradual and smooth, moderated by the microprocessor in a precise manor that is impossible to achieve by the traditional hand held syringe. The result is an “aim and shoot” technique.

According to one preferred embodiment, the anesthetic is delivered automatically at a rapidly increasing rate, which is programmed to accelerate from slow to rapid during a 5 second cycle. For example, the flow rate increases from 0 to 4.5 ml/min. Once at rapid speed, the higher flow rate is maintained until the injection is complete. The injection is administered without undue footwork, and with no unnecessary delay. An injection may take as little as 10 seconds which is about as fast as is capable by traditional hand held syringes. At any time during the injection, the dentist can interrupt the cycle by removing his/her foot from the foot pedal. This stops the injection. When the foot pedal is reactivated, the ramp cycle begins again from the beginning. The dentist may restart the cycle as often as desired to minimize patient discomfort. The dentist thus has the choice: a slow and gradual, series of ramps for total-pain control during the injection, or a rapid advance to high speed flow rate, to facilitate a rapid injection (as for example on a second injection when the patient is partially anesthetized and an increased dosage of the anesthetic solution is indicated). The illustrated “StandUp” portable injection device advantageously provides this total control of the flow rate, with the advantage of a pen-like syringe and easy visual monitoring by the dentist.

The illustrated “Standup” embodiment has a built in pressure sensor that indicates to the operator when optimal pressure is experienced. The “pressure sensor” feature is particularly helpful in achieving success in complicated injection procedures such as the peridontal ligament injection (PDL).

According to another aspect of this embodiment of the present invention, the existing microprocessor 18 or an alternative device is connected to monitor the current used by motor 14. As the load on the motor 14 increases the current drawn will also increase. This increase in current translates to an increase in the voltage which is also monitored and is compared to a fixed voltage set by a potentiometer. The output from the potentiometer is monitored by the microprocessor which can activate a discernable signal, such as a blinking red light when the load on the motor is in a predetermined range (corresponding to predetermined pressure range). If the motor encounters too much resistance, e.g., through a blockage in the flow of anesthetic, a second potentiometer senses a voltage overload, a signal is provided e.g. a constant red light, and the motor is turned off. The output from the second potentiometer is also monitored by the microprocessor which is designed to stop the motor.

The illustrated “StandUp” device can be used to administer all traditional infiltrations and block injections as well as injections which require pressure sensing.

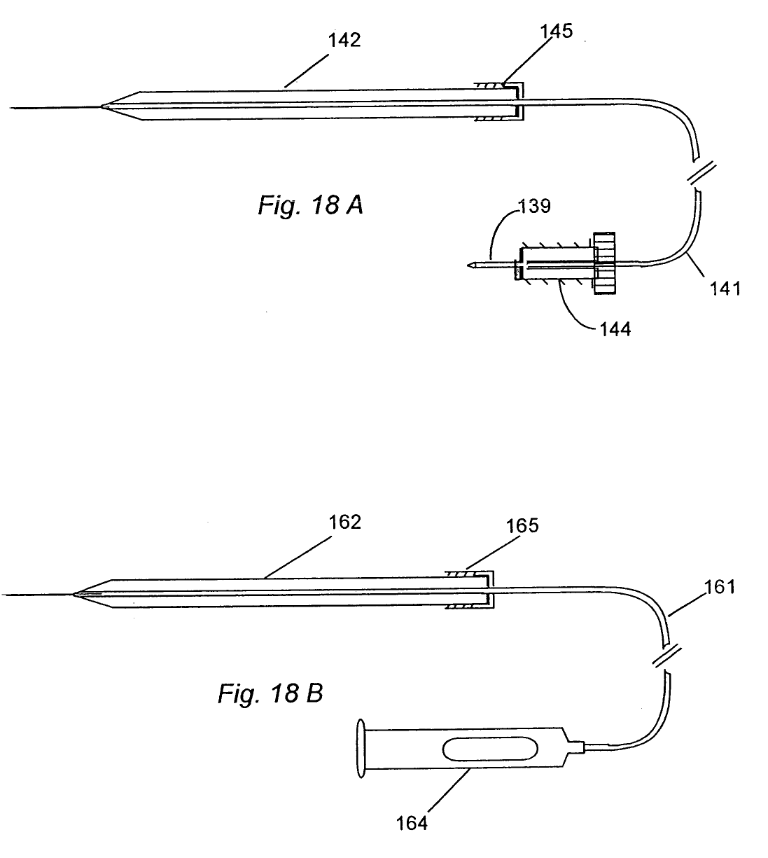

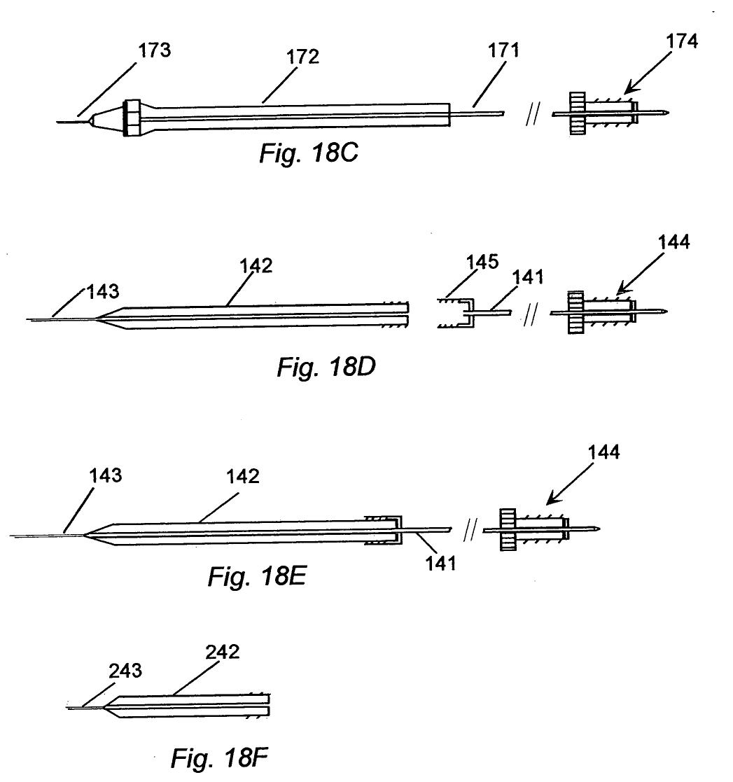

FIGS. 18(A)-(F) illustrate alternative embodiments of disposable syringe kits. In the embodiment shown in FIG. 18(A), a microbore tube 141 is provided with two threaded end caps. A proximal threaded cartridge connector 144 is designed to be threadably received within a cartridge holster 140 in a manner which causes a proximal needle 139 to puncture an anesthetic cartridge. As best shown in FIGS. 18(D) and 18(E), the distal end of the microbore tubing 141 is connected to a threaded distal needle handle connector 145 designed to be threadably connected to a disposable needle handle 142 which is attached to a needle 143. According to this embodiment of the present invention, the disposable needle handle can be formed of various lengths, as can the length of the needle which extends from the needle handle.

FIG. 18(F) illustrates a smaller needle handle 242 and smaller needle extension 243. If a dentist wishes to use a different size needle handle and different length needle on the same patient, according to this embodiment of the present invention, the dentist need not discard the entire syringe kit, but can simply replace the needle handle. The entire syringe kit need only be replaced when the dentist is finished treating that patient.

FIG. 18(B) illustrates an embodiment comprising a needle holster 164 at the proximal end of the flexible conduit 161 and a threaded distal connector 165 connected to a disposable needle handle 162.

FIG. 18(C) illustrates another embodiment of a disposable syringe kit comprising a proximal threaded cartridge connector 174 at the proximal end of a conduit 171 and a needle handle 172 fixed to the distal end of conduit 171. A needle 173 is removably attached to the distal end of the needle handle 172 with a luer lock attachment. Alternatively, the needle can be fixed to or integrally formed with the needle handle.

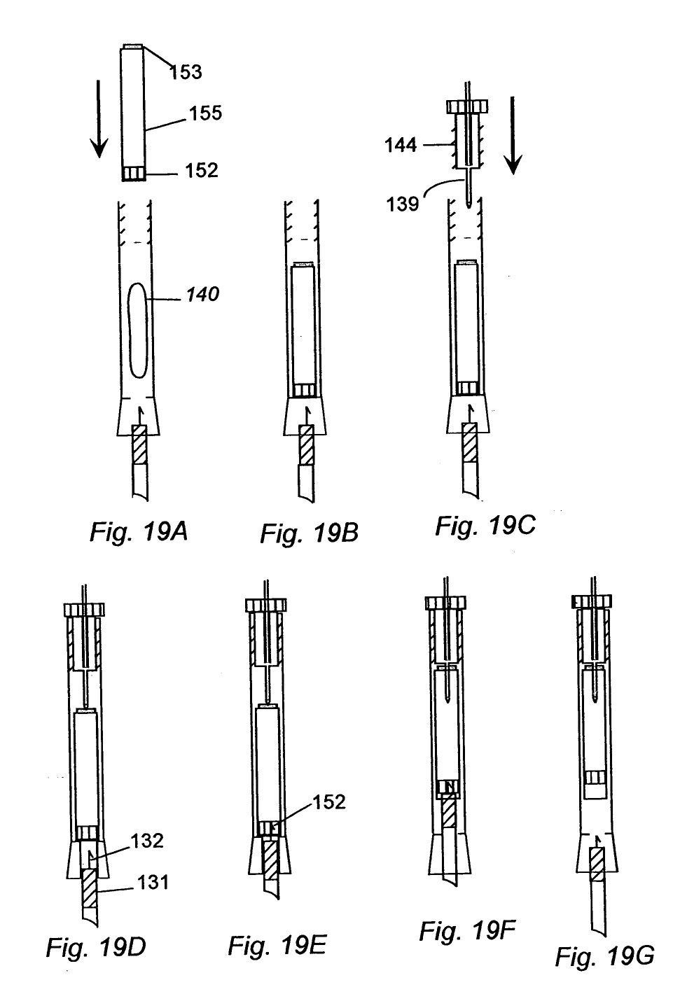

FIGS. 19(A) through (G) illustrate the positioning of a cartridge 155 in a delivery head according to this embodiment of the present invention. FIG. 19(A) illustrates the cartridge 155 being placed into a cartridge holster 140 as shown in FIGS. 19(A) and 19(B). Proximal threaded cartridge connector 144 is then positioned onto the top of the reciprocally threaded cartridge holster 140 as shown in FIGS. 19(C) and 19(D). When the plunger 131 is advanced, the barb 132 is caused to become seated within the cartridge piston 152 as shown in FIG. 19(E). The proximal needle 139 is designed so that it will not pierce membrane 153 of cartridge 155 until cartridge piston 152 has been fully seated on barb 132. Further advancement of plunger 131 will then cause proximal needle 139 to puncture cartridge membrane 153 as shown in FIG. 19(F). The cartridge is then ready for the dispensing/aspiration which occurs during a dental injection. After the injection is completed or the anesthetic has been depleted, the plunger 131 and barb 132 are simply withdrawn as shown in FIG. 19(G).

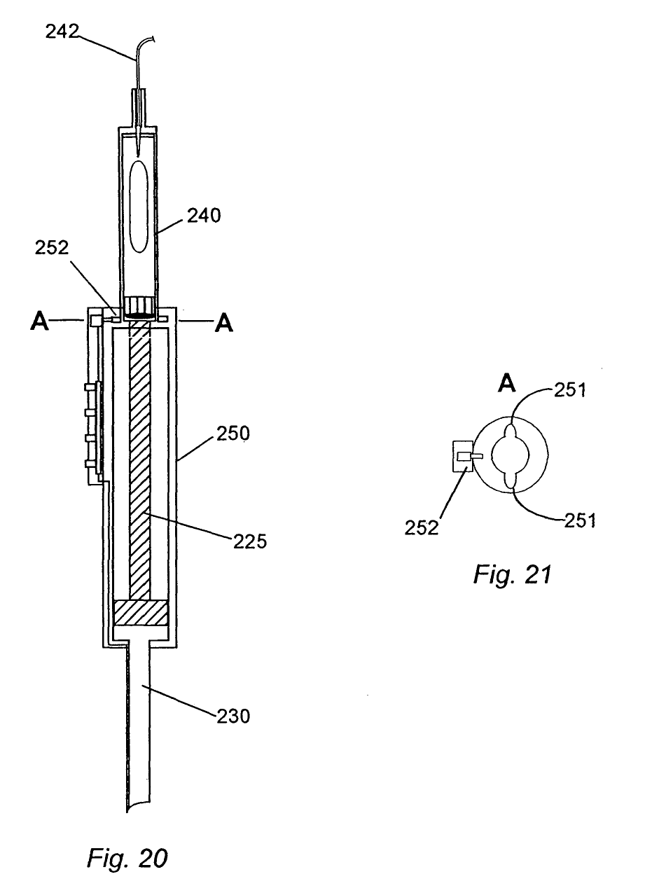

FIGS. 20 and 21 illustrate a further aspect of the present invention wherein FIG. 20 is a cross sectional side view and FIG. 21 is a cross sectional top view taken along lines A-A of FIG. 20. According to this embodiment of the present invention, needle holster 240 is formed with opposing tabs 241 on its proximal end which are designed to be received within corresponding tab openings 251 in cartridge support 250. When the cartridge holster 240 is positioned onto delivery head 250 and the cartridge holster 240 is rotated, cartridge holster tabs 241 engage a switch 252 which sends a signal resulting in the unit being powered and causing piston 255 to advance sufficiently to cause anesthetic fluid to enter tubing 242. According to the embodiment illustrated in FIG. 20, piston 255 in delivery head 250 is driven by hydraulic fluid 230. While this method of moving a piston to dispense anesthetic from the cartridge is less preferred than the rigid rod described above, from the present description, it will be understood that the present invention is not limited to the embodiment shown in FIG. 1.

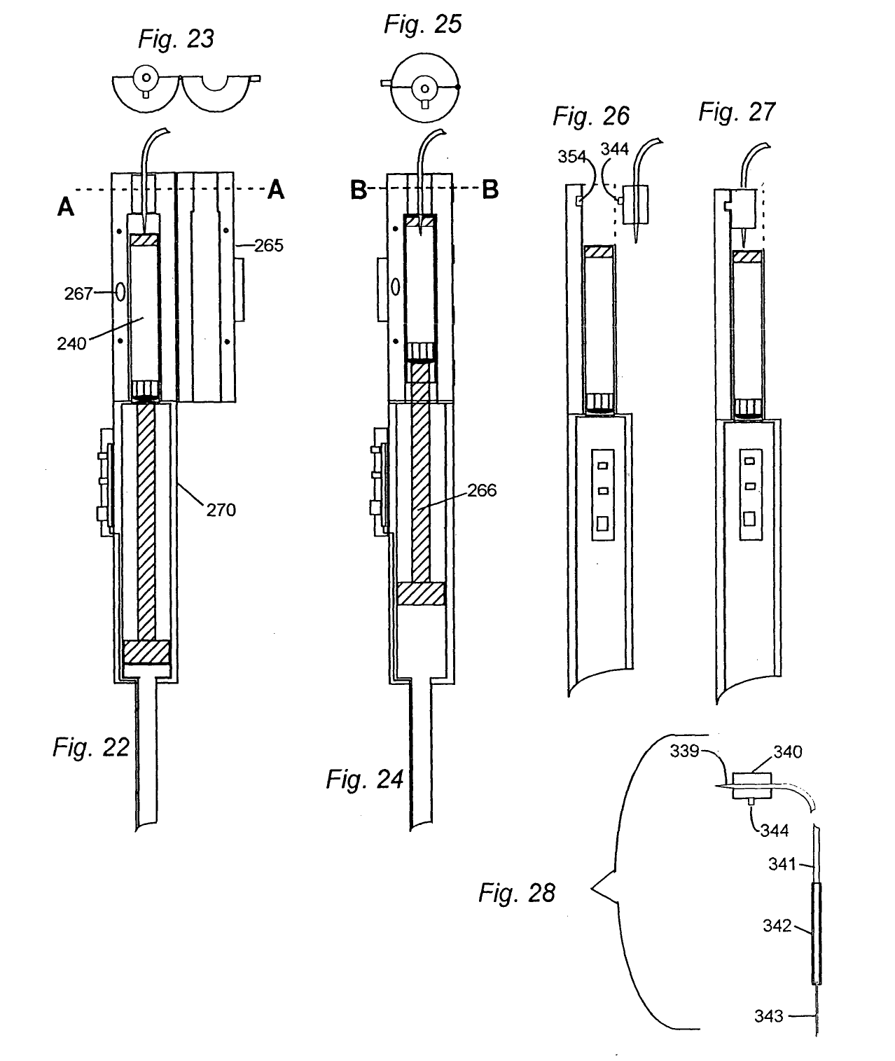

FIGS. 22-28 illustrate another embodiment of the present invention wherein an anesthetic cartridge is positioned within a delivery head 270 comprising an accessible and visible compartment. According to this embodiment of the present invention, the disposable syringe kit shown in FIG. 28 comprises a needle handle 342, a needle 343, tubing 341 and a connector block 340 and a proximal needle 339 as best shown in FIGS. 26 and 27, the connector block 340 comprises a positioning tab 344 which is positionable in a reciprocal slot 354 in the delivery head. According to this embodiment of the present invention, the delivery head comprises a compartment adapted to receive a cartridge 240 and the proximal-portion of the disposable syringe kit within a chamber having a hinged cover 265. According to this embodiment, cover 265 shown in FIG. 22 is opened, a cartridge 240 is positioned within the chamber as shown in FIG. 26 and then the proximal portion of the disposable syringe kit is also positioned in the chamber. When cover 265 is closed, a plunger 266 advances, preferably automatically upon the closing of the door in response to a switch 267 and the device is then ready for an injection.

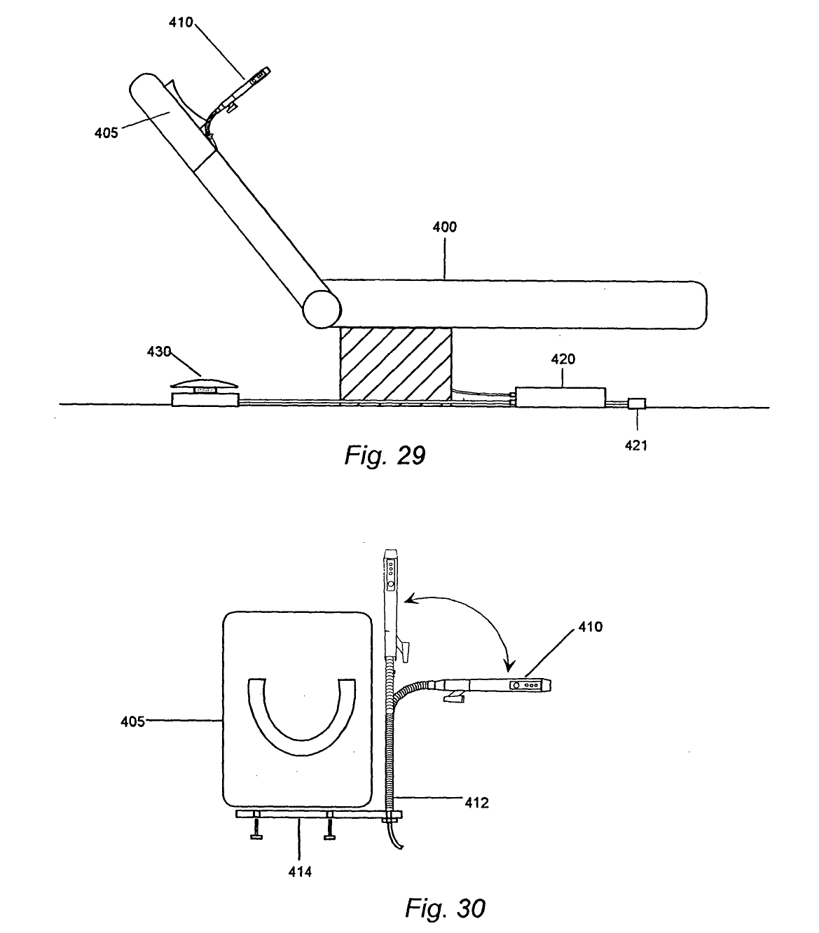

FIGS. 29 and 30 illustrate another embodiment of the present invention wherein a delivery head 410 is connected to a dental chair 400. According to this alternative embodiment, the delivery head 410 is connected to a gooseneck support 412 which is connected to the dental chair. In this illustrated embodiment, a bracket 414 connects the gooseneck support 412 to the headrest portion 405 of the chair 400. This arrangement advantageously enables a delivery head to be readily positioned out of the way when not in use and to be readily moved into position proximate the patient's mouth and within the dentist's field of vision for administration of an injection. FIG. 30 illustrates that the delivery head 410 can be rotated, for example, to a position in the same general plane as the headrest 405 of the dental chair 400. Either generally laterally or more toward a vertical position when not in use. According to this embodiment of the present invention, a control unit 420 is operated by a foot pedal 430 and receives power from a conventional A/C outlet 421. While this illustrated embodiment connects the delivery head to a dental chair, the delivery head can also be connected to other fixtures or equipment such as a bracket table, light, auxiliary table, or other parts of the dental unit. According to this embodiment, front pedal 430 is used to control a motor which is linked to a worm gear in control unit 420. This arrangement moves hydraulic fluid in a conduit in gooseneck 412. According to another embodiment, a flexible cable is used to connect the control unit 420 with a plunger in delivery head 410.

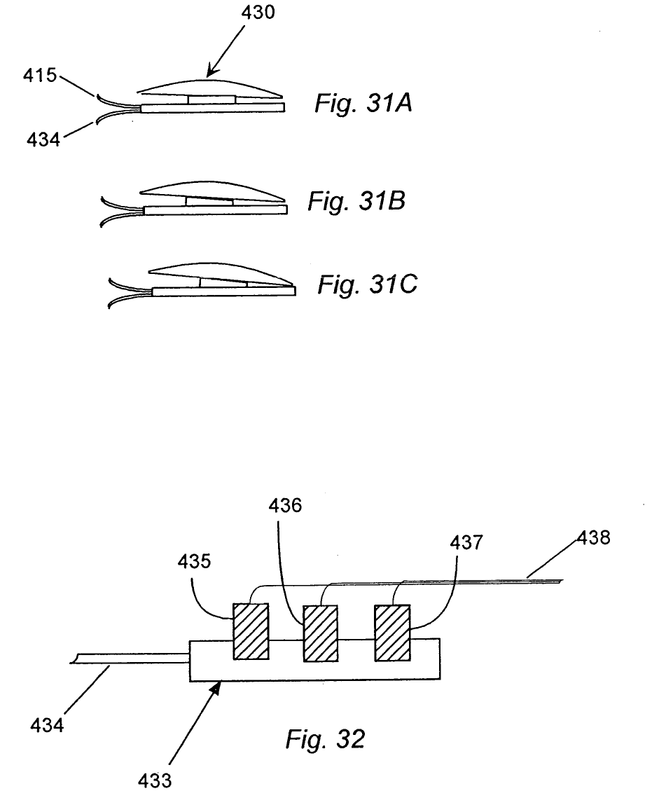

FIGS. 31(A)-(C) illustrate various positions of a dental food pedal useful with the present invention. This foot switch can advantageously be used with low voltage air pressure sensitive electric switches such as those shown in FIG. 32. FIG. 32 illustrates a manifold 432 connected to three air pressure sensitive electric switches. Air enters the manifold through a conduit 434. According to this embodiment, a food pedal switch such as those shown in FIG. 31 is preferably positioned between the air supply 415 and the manifold intake 434. Each of the air pressure sensitive switches 435, 436 and 437 are actuatable in response to different amounts of air pressure. Depression of a foot pedal slightly as shown in FIG. 31(A) is sufficient to permit about 10 psi of air pressure within manifold 433 which would consequently close switch 435 and send a signal to a motor or pump which is driving the plunger, to run at a slow speed. Further depression of the foot pedal to a position such as shown in FIG. 31(B) will permit more air pressure, for example, 40 psi to enter manifold 433. Thereby closing switch 436 which signals the motor to run in a middle speed range. Further depression of the switch to the point shown in FIG. 31(C) results in greater air pressure, for example, 60 psi, thereby closing switch 437 and resulting in the motor running at its fastest speed for rapid delivery of anesthetic. While the illustrated foot pedals are believed preferable, the advantage of the present invention can be utilized with other foot pedals.

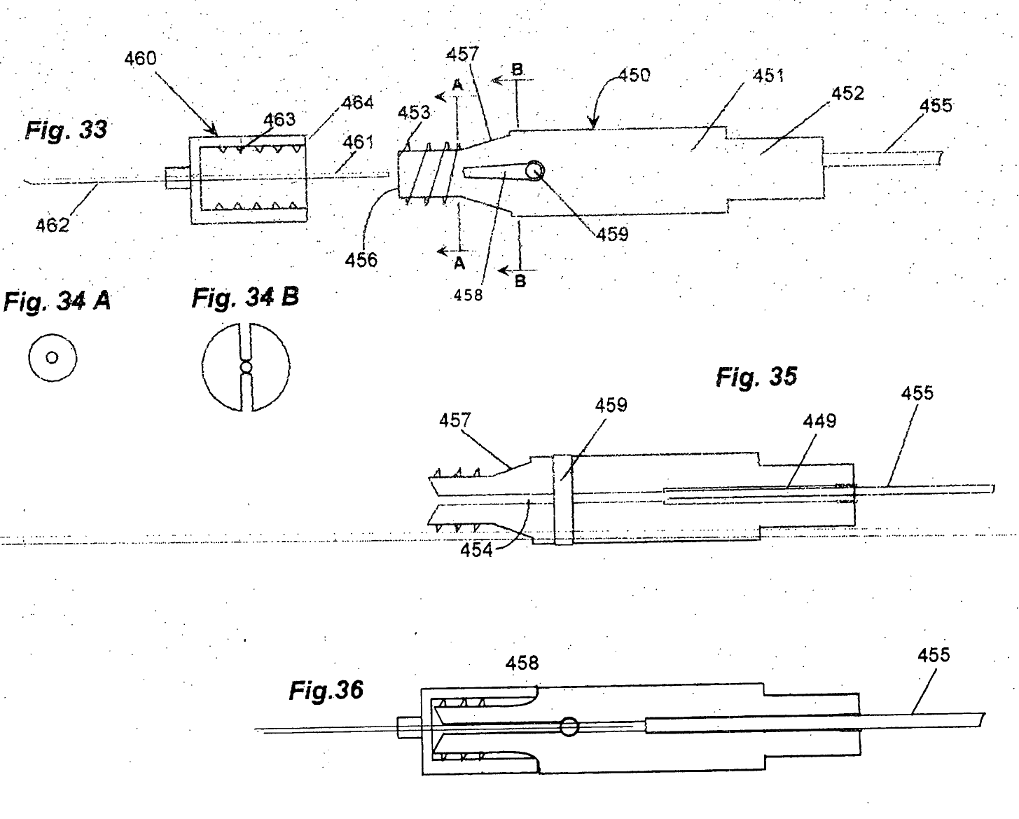

FIGS. 33-42 illustrate alternative needle handles of the present invention designed for use with known types of double-ended dental needles. FIG. 33 is a top view of a needle handle of the present invention. The illustrated handle 450 comprises a body section 451 and a proximal section 452. In this illustrated embodiment, the proximal section 452 is smaller than the body section and is designed for attachment to a flexible conduit 455, preferably microbore tubing. The microbore tubing can be connected to the proximal end of the needle handle 450 utilizing an adhesive or any other suitable method. In order to facilitate placement of conduit 455 in the reduced diameter proximal section 452 of needle handle 450, proximal portion 452 is preferably provided with a bore 449 dimensioned to receive conduit 455.

FIG. 33 also illustrates a conventional double-ended dental needle 460 comprising a needle having a proximal end 461, a distal end 462 and a threaded connector 463 having a proximal end 464. Connector 463 is securely attached to the needle. Needle handle 450 comprises a distal end 453 having a distal bore 454. Distal bore 454 guides a needle into the needle handle 450 in a manner described in greater detail below to establish fluidic communication between flexible conduit 455 and the needle.

According to this illustrated embodiment, the distal section 453 of needle handle 450 comprises a threaded exterior surface 456 and a resilient sloped surface 457. Needle handle 450 also comprises a pair of tapered slots 458 and a seal 459. Seal 459 is preferably formed of a resilient material, for example, a rubber-like material or other materials suitable for providing a fluid-tight seal between seal 459 and needle 461, as well as between seal 459 and the adjacent portion of needle handle 450. The seal 459 can be held in the handle 450 with an interference fit, with an adhesive such as cyanoacrylate, by the structure of the needle handle, or in any other suitable manner. Needle handle 450 is designed to provide a leak resistant and selectively removable attachment with a double-ended needle handle.

According to this preferred embodiment of the present invention, in order to connect a double-ended needle to the needle handle 450, the proximal end of needle 461 is slid into distal bore 454 of the needle handle. According to this preferred embodiment, the distal bore 454 extends from the distal end of needle handle 450 through the threaded connecting portion 453 of needle handle and through the tapered, slotted section 457 as illustrated in the cross-sectional views in FIGS. 34A and 34B, to a point proximate seal 459. Either before or as the double-ended dental needle 460 is being screwed onto the distal end of needle handle 450, the proximal end 461 of the dental needle passes through seal 459 which forms a fluid-tight seal around the needle. This establishes fluidic communication between the needle and flexible conduit 455.

During the connecting of the double-ended needle 460 with the needle handle 450, proximal end 464 of attachment section 463 contacts resilient sloped surface 457 and slightly compresses this portion of the needle handle 450 as well as the portion adjacent to seal 459. The compression of the portion of the needle handle 450 proximate the seal 459 tightens the seal and diminishes the likelihood of leaks during an injection.

FIG. 36 illustrates the double-ended dental needle 460 secured to the needle handle 450 with the proximal end 461 of the needle passing through the seal 459 and the conduit 455 in fluidic communication with the needle.

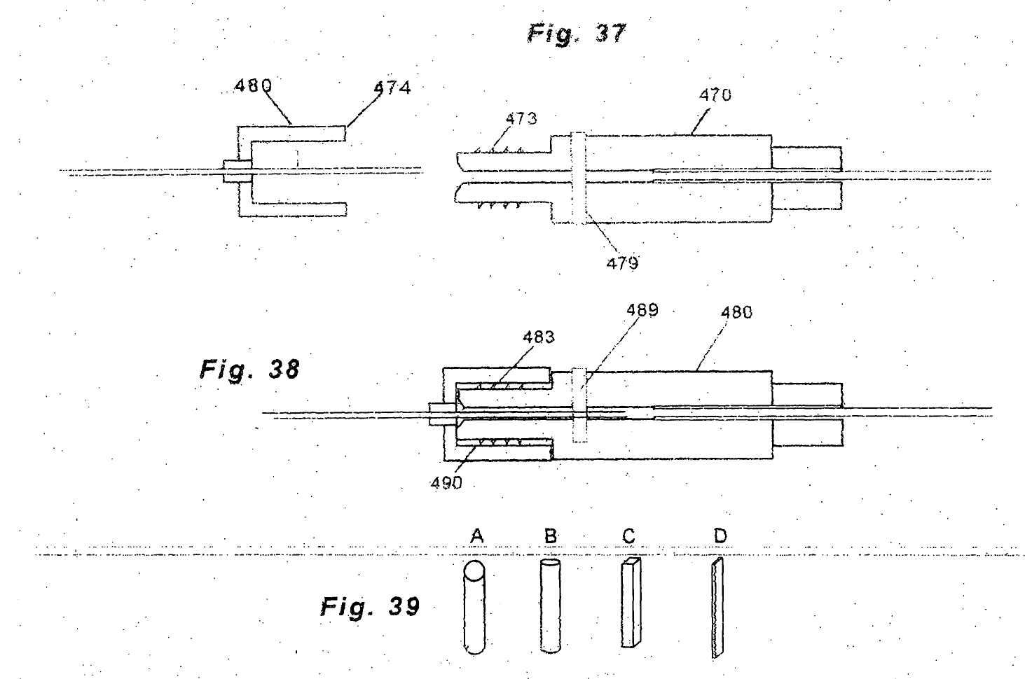

FIGS. 37 and 38 illustrate a side view and an assembled view, respectively, of alternative embodiments of the present invention. The embodiment of FIG. 37 comprises a seal 479 having the general shape of a square. In this embodiment, the seal is secured within a through hole in the handle utilizing an adhesive such as cyanoacrylate which is preferably soluble in at least one of the compounds used in common local dental anesthetic solutions. According to the embodiment of the present invention illustrated in FIG. 38, the illustrated seal 489 does not extend entirely through handle 480. According to these embodiments of the present invention, the needle handle does not have a ramped surface for engagement with the proximal end of double-ended dental needle. FIGS. 37 and 38 also illustrate a standard double-ended dental needle 470 which is not prethreaded but which is self threaded on threads 473 on the distal end of needle handle 470.

FIGS. 39A-D illustrate other seals having different shapes which can be used with the present invention. The shapes illustrated in FIGS. 39A-D are round, ovoid, rectangular and flat, respectively. From the present description, those skilled in the art will appreciate that seals having other shapes and/or sizes can be used without departing from the scope of the present invention.

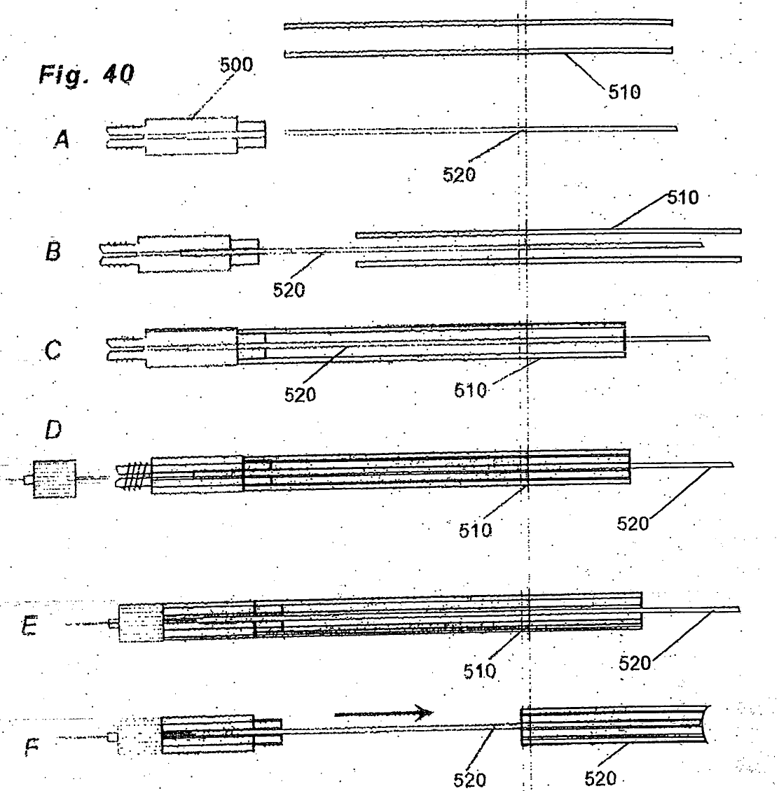

FIGS. 40A-F illustrate another aspect of the present invention wherein a needle handle 500 is provided with a handle extension 510. According to the this preferred embodiment of the present invention, a handle extension 510 is generally formed as a hollow cylinder which has an interior size and shape adapted for an interference fit over a corresponding proximal section of needle handle 500. As used herein, the term “interference fit” is used to indicate that two pieces are dimensioned and/or configured so that there is sufficient friction between two relatively movable parts that they will normally remain together, but can be separated when desired.

FIG. 40A illustrates the needle handle separate and apart from the handle extension 510 prior to attachment of the conduit 520 to the needle handle 500. FIG. 40B shows the handle extension 510 positioned around the conduit 520 which is attached to needle handle 500. FIG. 40C shows the handle extension 510 positioned on needle handle 500. FIGS. 40D and 40E illustrate the attachment of a double-ended needle to the needle handle having the extension. FIG. 40E illustrates the assembled needle handle with both the extension 510 and the double-ended needle attached. In this configuration, the dentist can administer anesthesia if a full size handle is desired. FIG. 40F illustrates how the handle extension 510 can be slid back on the conduit 520 if and when a dentist prefers to have a smaller needle handle. The handle extension 510 can preferably be slid back the entire length of the conduit 520. If desired, structure can be provided proximate the proximal end of the flexible conduit 520 in order to secure the handle extension 510 in a position which will not interfere with the dental procedure.

While the handle extension illustrated in FIGS. 40E-F has a generally circular cross-section, and is attached to the body of the needle handle with a simple interference fit, it is within the scope of the present invention to form handles and/or extensions with different shapes, sizes and with different connections between the needle handle and the extension, for example, snap-fits, threaded connections, detents/recesses, etc.

The needle handles of the present invention are preferably formed of a polymeric material such as PVC, polyethylene, polypropylene or other materials known to those in the art. The seals are preferably formed of a natural or synthetic rubber or other rubber-like or resilient material capable of forming a fluid tight seal around the exterior of a needle passing through the seal as well as between the seal and the needle handle.

According to one embodiment of the present invention, the adhesive used to maintain the seal within the needle handle is at least partially soluble in a common solution of local dental anesthetic. Thus, while the seal is designed to function properly during use of a single needle, if a dental technician is tempted to continuously reuse a needle handle of this embodiment of the present invention, the solubility of the adhesive in the dental solution will degrade the integrity of the fluid-tight seal between the seal and the needle handle resulting in leakage. This will tend to discourage repeated uses. This planned obsolescence is designed to discourage unsanitary repeated uses of a single needle handle with different dental patients.

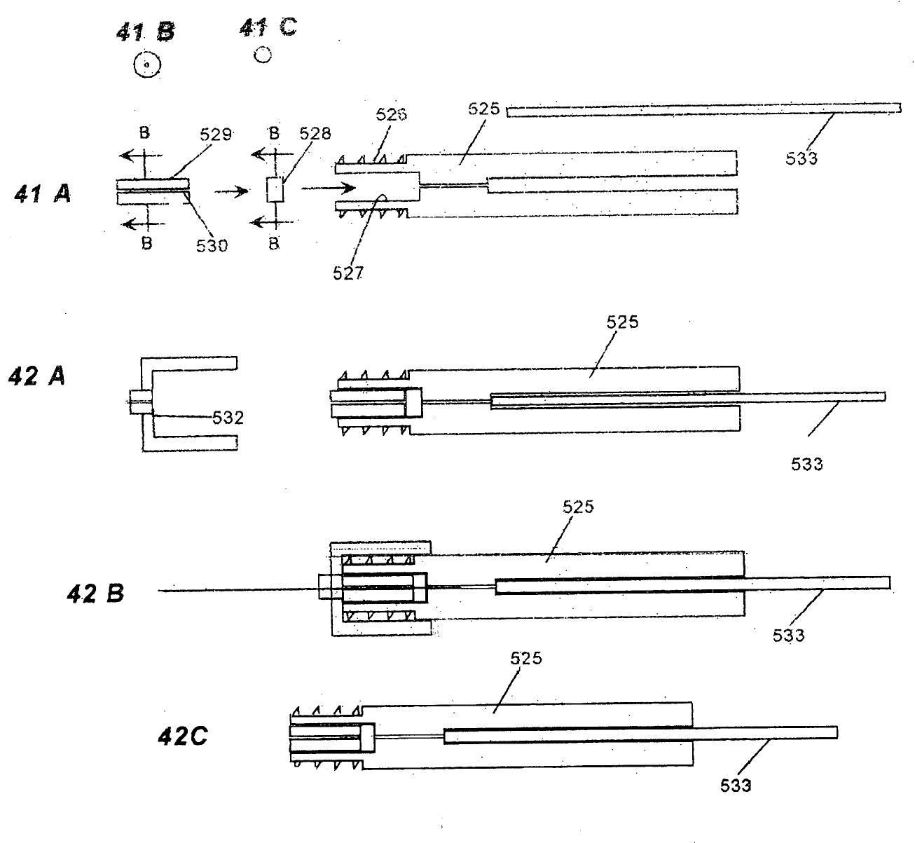

According to another embodiment of the present invention illustrated in FIGS. 41 and 42, the distal end of a needle handle 525 is provided with a connector portion 526 and a seal recess 527. According to this illustrated embodiment, a seal 528 is inserted into seal recess 527 and then a guide 529, for example formed of a polymer material, having an internal bore 530 is inserted recess 527. The seal 528 and the guide 529 can be dimensioned to form an interference fit with recess 527. This arrangement facilitates assembly and reduces assembly costs. In this manner, when a double ended dental needle 535 is threaded onto the threaded connections 526 on the distal end of the needle handle 525, the proximal side of base 532 of needle 535 will force the guide 529 further into recess 527 thereby compressing the seal 528. This compression enhances the fluid tight seal between the needle and seal 528, as well as between the seal 528 and needle handle 525 while providing fluidic communication between the needle and the flexible conduit 533. FIG. 42A illustrates a double ended needle ready to be attached to needle handle 525. FIG. 42B illustrates the double ended needle of FIG. 42A attached to needle handle 525. According to another embodiment of the present invention shown in FIG. 42C, the seal 528 and guide 539 are secured within the distal recess 537, for example with an adhesive.

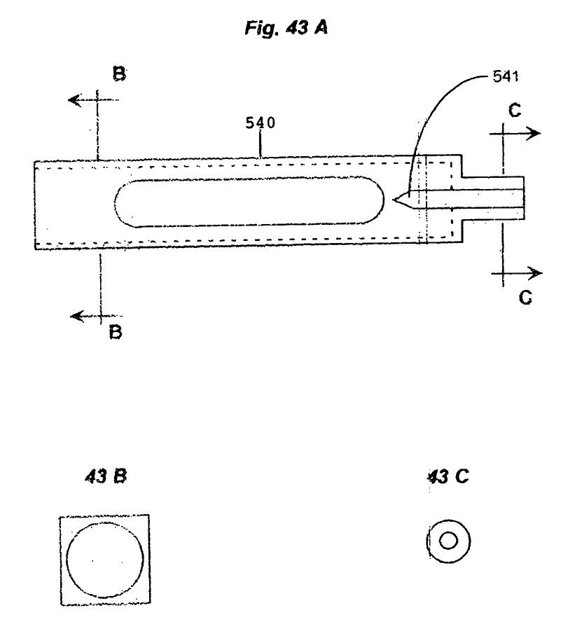

FIGS. 43A-C illustrate an alternative cartridge holster of the present invention having a square cross section. This cartridge holster is connected to the proximal end of a flexible conduit (not shown) and comprises a generally hollow body 540 having an internal cavity adapted to receive a disposable cartridge of dental anesthetic and a hollow sharpened piercing member 541 for piercing the membrane of the disposable dental anesthetic cartridge.

As best shown in FIG. 43B (cross section taken along lines B-B), the exterior of the body of the cartridge holster is preferably square while a neck portion to which is attached conduit 530 can be generally circular as shown in FIG. 43C.

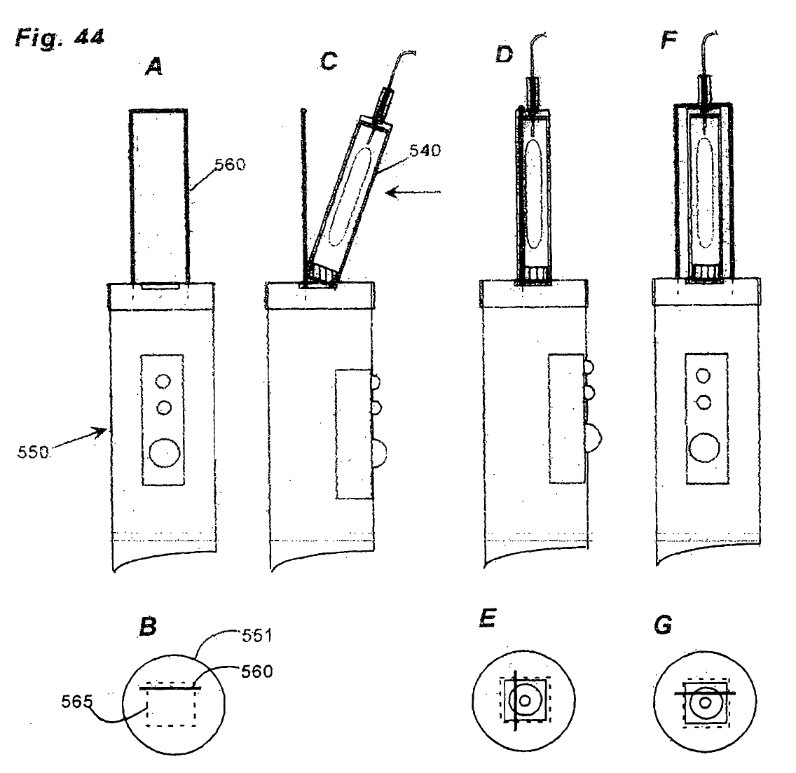

With reference to FIGS. 44A-G, the cartridge holster 540 shown in FIGS. 44A-C is designed for attachment to a delivery head 550 by placing the holster 540 along with a disposable cartridge of local dental anesthetic under bracket 560 which extends upwardly from the delivery head 550. Bracket 560 is preferably formed of a coated wire which is securely connected to delivery head 550. Alternative materials for bracket 560 include stainless steel, plastic, reinforced plastic, other metals, and combinations thereof. Delivery head 550 can be formed with a recess to receive a proximal portion of the cartridge holster 540. In the manner illustrated in FIGS. 44A-G, the base of a cartridge holster is placed into the delivery head in a manner which allows a substantial portion of the holster to be positioned under bracket 560 thereby securely maintaining cartridge holster 560 along with the anesthetic cartridge securely on the delivery head during the dispensing of the anesthetic solution. FIG. 44A is a front view of a delivery head comprising bracket 560. FIG. 44B is a top view illustrating the external surface 551 of delivery head 550, the top of bracket 560 and the outline of the recess 565. FIG. 44C illustrates the initial placement of a portion of the cartridge holster 540 onto delivery head 550. FIGS. 44D-G are a side view, top side view, front view and top front view, respectively, of the cartridge holster 540 securely maintained onto delivery head 550 by bracket 560.

As noted above, this bracket is designed to securely maintain the cartridge holster including a cartridge of dental anesthetic onto the top of the delivery head. It is particularly desirable to provide a secure connection during high pressure injections such as PDL injections.

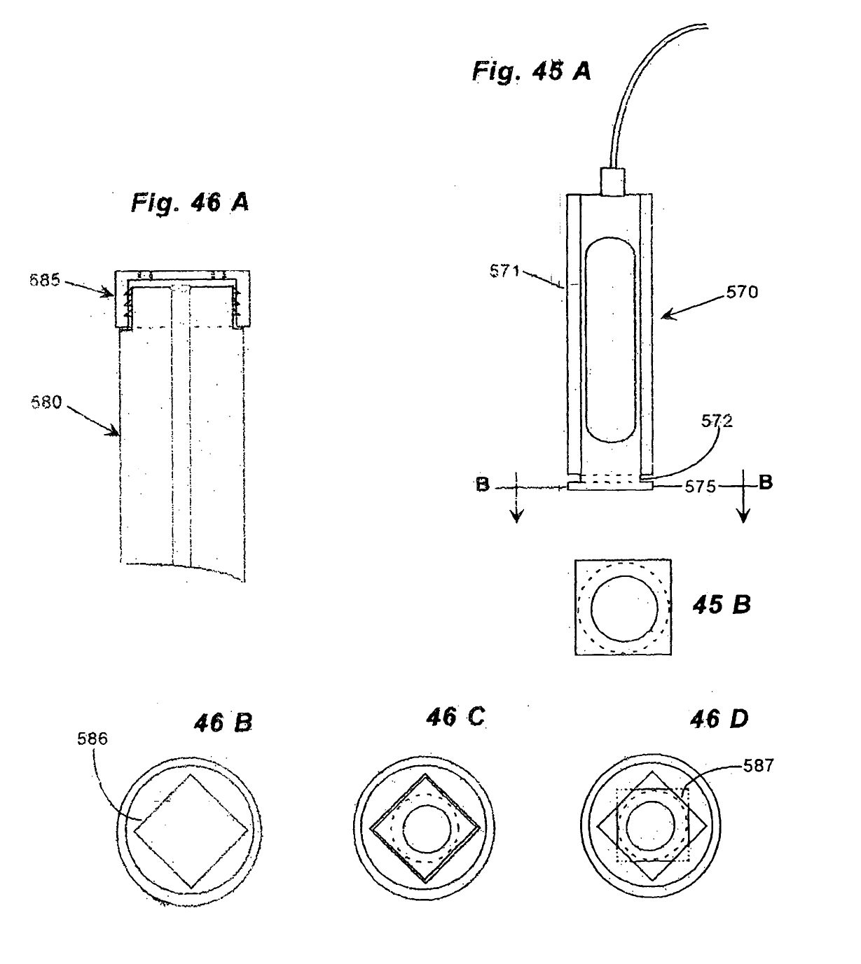

FIGS. 45A-B and 46A-D illustrate alternative embodiments. In the embodiment of FIG. 45, cartridge holster 570 comprises a base 575 separated from holster body 571 by a neck 572 which has an external surface smaller than the external surface of holster body 571. As illustrated in FIG. 45B, the holster base 575 of this illustrated embodiment of the present invention has a generally square cross section and is adapted to be rotatably received within delivery head 580. The upper portion 585 of delivery head 580 comprises a square recess 586 into which holster base 575 is positioned. Upper portion 585 of delivery head 580 also comprises a plurality of recesses corresponding to each of the corners of holster base 575 which permit rotation of cartridge holster 570 through an angle of about 30°-70° on delivery head thereby locking base 575 under rims 587 of upper portion 585.

I claim:

1. A disposable dental syringe kit, for use with a double ended dental needle comprising a needle extending through an attachment portion, said kit comprising:
a flexible conduit comprising a proximal end and a distal end;
a needle handle connected to said flexible conduit proximate said distal end,
said needle handle comprising a distal bore for receiving the proximate end of a double ended needle, a connector for connecting an attachment portion of a double ended needle to the needle handle thereby placing the flexible conduit in fluidic communication with a needle, and a pierceable seal through which a needle is placed when connecting a double ended needle to said needle handle.

2. A disposable syringe kit according to claim 1 wherein said seal comprises a rubber-like material.

3. A disposable syringe kit according to claim 2 wherein said seal is generally cylindrical.

4. A disposable syringe kit according to claim 2 wherein said seal is generally ovoid.

5. A disposable syringe kit according to claim 2 wherein said seal is generally rectangular.

6. A disposable syringe kit according to claim 2 wherein said seal is generally flat.

7. A disposable dental syringe kit according to claim 2 wherein said seal is snugly received in a recess in said needle handle which forms an interference fit with said seal.

8. A disposable syringe kit according to claim 3 wherein said seal is secured to said needle handle with an adhesive.

9. A disposable syringe kit according to claim 8 wherein said adhesive is soluble in dental anesthetic.

10. A disposable syringe kit according to claim 1 wherein said seal is generally cylindrical.

11. A disposable syringe kit according to claim 1 wherein said seal is generally ovoid.

12. A disposable syringe kit according to claim 1 wherein said seal is generally rectangular.

13. A disposable syringe kit according to claim 1 wherein said seal is generally flat.

14. A disposable syringe kit according to claim 1 wherein said seal is secured to said needle handle with an adhesive.

15. A disposable syringe kit according to claim 8 wherein said adhesive is soluble in local dental anesthetic.

16. A disposable syringe kit according to claim 1 wherein said connector comprises a resilient sloped surface which is contacted by an attachment portion of a double ended needle during attachment of a double ended needle to said needle handle.

17. A disposable syringe kit according to claim 16 wherein at least a portion of said needle handle is compressed during attachment of a double ended needle to said needle handle.

18. A disposable syringe kit according to claim 16 wherein a portion of said needle handle adjacent said seal is compressed during attachment of a double ended needle to said needle handle.

19. A disposable syringe kit according to claim 16 comprising at least one recess adjacent said seal.

20. A disposable syringe kit according to claim 19 comprising at least two recesses adjacent said seal.

21. A disposable syringe kit according to claim 19 wherein said seal is located in at least a portion of said recess.

22. A disposable syringe kit according to claim 1 wherein said distal bore extends proximally through said needle handle at least to a point proximal of the distal-most portion of said recess.

23. A disposable syringe kit according to claim 1 wherein said connector comprises threads.

24. A disposable syringe kit according to claim 1 further comprising a handle extension removably attached to said needle handle.

25. A disposable syringe kit according to claim 1 comprising a guide comprising a bore, said guide disposed distally of said seal.

26. A disposable syringe kit according to claim 1 wherein said guide is removable positioned at least partially in said needle handle.

27. A disposable syringe kit according to claim 1 wherein said guide is removably positioned with an interference fit.

28. A disposable syringe kit according to claim 1 wherein said guide is secured to said needle handle.

29. A disposable syringe kit according to claim 1 wherein said guide is secured to said needle handle with an adhesive.

30. A disposable dental syringe kit comprising:
a flexible conduit comprising a proximal end and a distal end;
a needle handle connected to a said flexible conduit proximal said distal end, and
a handle extension removably attached to said needle handle.

31. A disposable dental syringe kit according to claim 30 wherein said handle extension is removably connected to a proximal portion of said needle handle.

32. A disposable dental syringe kit according to claim 30 wherein said handle extension is threadably connected to said needle handle.

33. A disposable dental syringe kit according to claim 30 wherein said handle extension is connected to said needle handle by sliding said handle extension over a portion of said needle handle.

34. A disposable dental syringe kit according to claim 30 wherein said handle extension comprises a hollow portion which forms an interference fit with a portion of said needle handle.

35. A disposable dental syringe kit according to claim 30 wherein said needle handle comprises a hollow portion which forms an interference fit with a portion of said handle extension.

36. A dental injection apparatus comprising:
a delivery head for pumping anesthetic from a disposable cartridge of dental anesthetic, said cartridge comprising a piston and said delivery head comprising a movable plunger for moving said piston;
a cartridge holster adapted to receive a disposable cartridge of dental anesthetic,
said delivery head comprising a bracket for securely and releasably connecting said cartridge holster to said delivery head.

37. A dental injection apparatus according to claim 36 wherein at least a portion of said bracket extends a sufficient distance from said plunger so that a major portion of said cartridge holster can be positioned substantially between a portion of said bracket and said plunger prior to pumping.

38. A dental injection apparatus according to claim 36 wherein said bracket comprises a wire.

39. A dental injection apparatus according to claim 38 wherein said wire is a coated wire.

40. A dental injection apparatus according to claim 38 wherein said bracket comprises a metal.

41. A dental injection apparatus according to claim 38 wherein said bracket comprises stainless steel.

42. A dental injection apparatus according to claim 38 wherein said bracket comprises a polymeric material.

43. A dental injection apparatus according to claim 38 wherein said bracket comprises a reinforced polymeric material.

44. A dental injection apparatus comprising:
a delivery head for pumping anesthetic from a disposable cartridge of dental anesthetic, said cartridge comprising a piston and said delivery head comprising a movable plunger for moving said piston;
a cartridge holster adapted to receive a disposable cartridge of dental anesthetic,
said cartridge holster comprising a base with an outer surface comprising a polygonal cross section;
said delivery head comprising a recess for securely and releasably connecting said holster base to said delivery head.

45. A dental injection apparatus according to claim 44 wherein said outer surface of said holster comprises a generally square cross section and said deliver head comprises a square opening and a plurality of slots which engage a plurality of the corners of said holster base to securely and releasably connect said holster to said delivery head.
